(19) United States
(12) Patent Application Publication
(10) Pub. No.: US 2007/0161962 A1
(43) Pub. Date: Jul. 12, 2007

(54) Device and method for moving fill material to an implant

(76) Inventors: Jason A. Edie, Memphis, TN (US); John White, Bartlett, TN (US); James Douglas Schwender, Edina, MN (US)

Correspondence Address: COATS & BENNETT, PLLC, 1400 Crescent Green, Suite 300, Cary, NC, 27518, US

(21) Appl. No.: 11/327,888

(22) Filed: Jan. 9, 2006

(51) Int. Cl. A61F 2/46; A61F 2/44

(52) U.S. Cl. 604/257; 623/17.12

(57) ABSTRACT

Devices and methods for inserting an implant within a patient. The device may generally include an insertion body, a supply reservoir, and a delivery device. The insertion body can be used for moving and positioning the implant within the body. In one embodiment, implant is mounted to the end of the body prior to movement and positioning. Fill material is maintained within the supply reservoir. Delivery device functions to move the fill material from the supply reservoir, through the insertion body, and to the implant. The device thus provides for both movement and placement of the implant, and a mechanism for delivering fill material to the implant.

### BACKGROUND

Implants are inserted into the body for a variety of applications. One application is an implant positionable between adjacent vertebral members. The implant includes a first surface that is positioned against a first vertebral member, and a second surface that is positioned against a second vertebral member. Once positioned, the implant may be fixedly positioned in the body to accurately space apart the vertebral members.

Because the implants are often positioned within relatively small areas of the body, various tools are necessary for accurately placing the implant. The tools may include movable jaws for grasping and placing the implant within the body. Once placed, the jaws are movable to an open orientation for releasing the implant. The tools may have an elongated shape such that the surgeon can remain distant with only the tool and implant being placed within the patient.

The implants may include a receiving section for receiving fill material. The receiving section may be fixed having a permanent size established by the structure or implant, or may have an expandable size that enlarges upon insertion of the fill material. The receiving section is usually positioned in a manner that is not easily accessible once the implant is inserted within the body. As a result, the normal procedure requires the receiving section to be filled with fill material prior to insertion.

### SUMMARY

The present application is directed to devices and methods for inserting an implant within a patient. The device generally includes an insertion body, a supply reservoir, and a delivery device. The insertion body is used for moving and positioning the implant within the body. In one embodiment, the implant is mounted to the end of the body prior to movement and positioning. Fill material is maintained within the supply reservoir. The delivery device moves the fill material from the supply reservoir, through the insertion body, and to the implant. The device thus provides for both movement and placement of the implant, and a means for delivering fill material to the implant.

### BRIEF DESCRIPTION OF THE DRAWINGS

FIG. 1 is a schematic diagram of an insertion device and an implant according to one embodiment.

FIG. 2 is schematic diagram of an insertion device and an implant according to one embodiment.

FIG. 3 is a schematic diagram of an implant and partial view of the distal end of the insertion body according to one embodiment.

FIG. 4 is a schematic diagram of an insertion device and implant according to one embodiment.

FIG. 5 is a schematic diagram of an insertion device and implant according to one embodiment.

### DETAILED DESCRIPTION

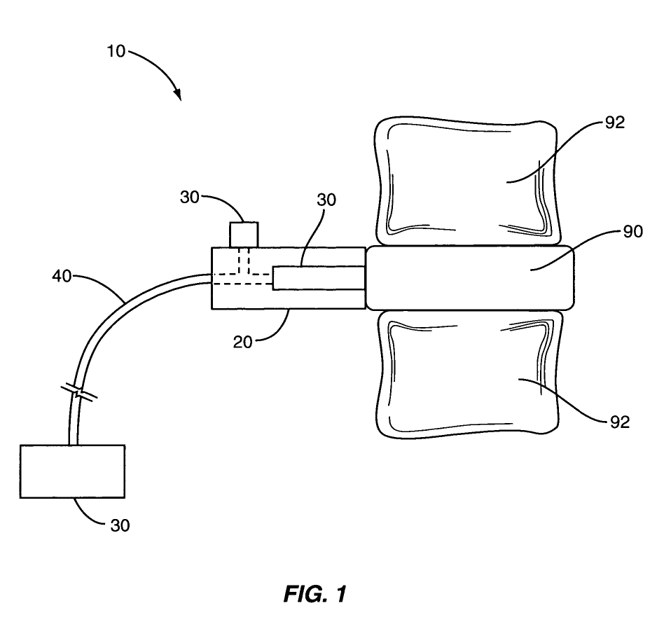

An insertion device, generally illustrated as element 10 in FIG. 1, for positioning an implant 90 within a body. The insertion device 10 further provides a conduit for inserting fill material within the interior of the implant 90 after it has been positioned within the body between vertebral members 92. In the embodiment of FIG. 1, the insertion device 10 comprises an insertion body 20 that contacts the implant 90. A supply reservoir 30 is operatively connected to the insertion body 20. A delivery device 40 moves the fill material from the supply reservoir 30 and through the insertion body 20 for insertion into the implant 90.

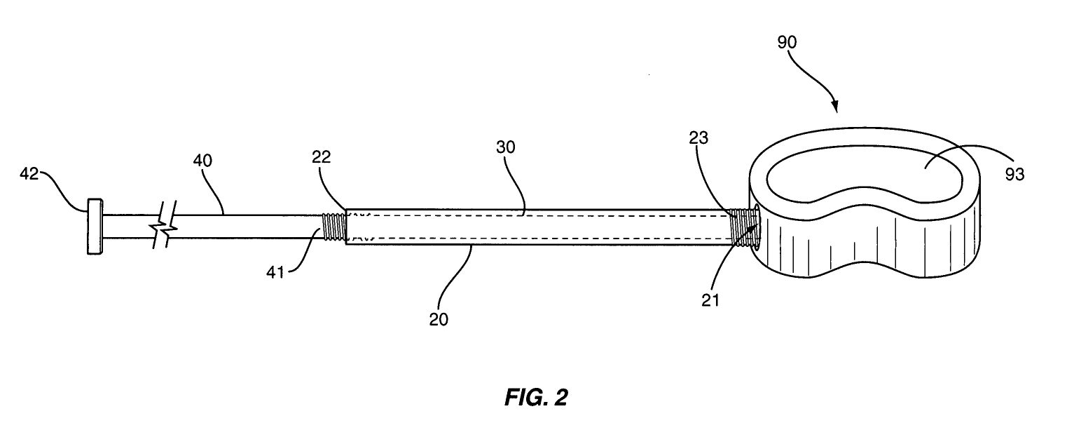

In one embodiment, the insertion body 20 includes a distal section 21 and a proximal section 22 as illustrated in FIG. 2. The insertion body 20 is rigid thus providing a mechanism for moving and positioning the implant 90 within the body. Further, insertion body 20 may have an extended size such that the distal section 21 is positioned to place the implant 90 within the body, while the proximal section 22 is positioned outside of the body and can be more easily manipulated by the surgeon. Insertion body 20 is hollow providing a conduit for moving the fill material to the implant 90. In the embodiment of FIG. 2, the hollow section forms the supply reservoir 30 for storing the fill material. The distal section 21 may include a connection mechanism 23 for attachment to the implant 90. Connection mechanism 23 may include threads that mate with corresponding threads in the implant 90.

In one embodiment, delivery device 40 includes a plunger having a first end 41 and a second end 42. First end 41 is sized to mate with the proximal section 22 of the insertion body 20. Insertion of the first end 41 into the insertion body 20 forces the fill material within the supply reservoir 30 to be expelled from the distal section 21 and into the implant 90. In one embodiment, a section or the entirety of the supply reservoir 30 is threaded to receive the threaded plunger 42. Rotation of the plunger 42 moves the plunger through the reservoir 30 thus expelling fill material from the distal end 21. In another embodiment, an axial force is applied to the delivery device 40 thereby moving the first end 41 through the supply reservoir 30 and expelling fill material from the distal end 21. The second end 42 may include an enlarged surface for receiving an impact force for driving the delivery device 40 through the insertion body 20.

The term “fill material” is used in a general sense throughout to describe osteogenetic material that is inserted into and/or adjacent to the implant 90. The fill material may include autograft, allograft, xenograft, demineralized bone, synthetic and natural bone graft substitutes, such as bioceramics and polymers, and osteoconductive and osteoinductive factors. The fill material has a composition that permits movement from the supply reservoir 30 through the insertion body 20 and to the implant 90.

Use of the device described in FIG. 2 comprises initially placing fill material within the supply reservoir 30 prior to beginning the procedure. Implant 90 is then either connected to the distal end 21, or positioned within the body with the distal end 21. Prior to placement of the implant 90, the delivery device 40 may be mounted to the insertion body 20. This increases the overall length of the device 10 and facilitates placement and/or movement of the implant 90 by allowing the surgeon to grasp the second end 42 of the delivery device 40 and manipulate the implant 90. The rigidity of the insertion body 20 and the delivery device 40 allows the surgeon to use these elements to accurately locate the implant 90.

After the implant 90 has been accurately located, the delivery device 40 is moved through all or a section of the supply reservoir 30 to force the fill material to the implant 90. The amount of fill material moved to the implant 90 may vary depending upon the size of the implant 90 and the specific application. The surgeon can tactilely feel the movement of the delivery device 40 moving through the insertion body 20 to roughly control the amount of fill material expelled through the distal end 21. Gauge lines on the delivery device 40 and/or insertion body 20 may also provide visual confirmation regarding the amount of expelled fill material. Once the fill material is moved to the implant 90, the insertion body 20 and delivery device 40 are removed from the body. The implant 90 with fill material then remains permanently positioned within the patient.

A spacer (not illustrated) may be positioned within the supply reservoir 30 between the delivery device 40 and the fill material. Spacer fills a portion of the supply reservoir 40 and limits the amount of needed fill material. The delivery device 40 acts on the spacer which in turn acts on the fill material to expel it from the distal end 21.

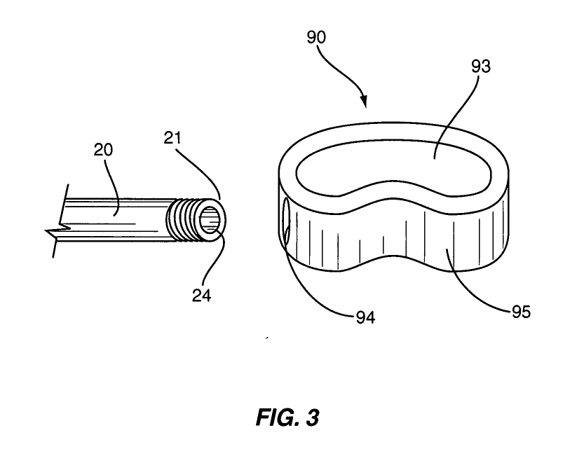

FIG. 3 illustrates one embodiment of the implant 90 and distal end 21. Implant 90 includes a hollow interior section 93 formed by a rigid exterior shell 95. An aperture 94 through the shell 95 leads into the interior section 93. Positioning the implant 90 between vertebral members 92 causes access to the interior section 93 to be limited to the aperture 94. Distal section 21 of the insertion body 20 is sized to fit within the aperture 94. The distal section 21 includes an opening 24 that communicates with the supply reservoir 30. Movement of the fill material through the insertion body 20 causes expulsion through the opening 24 and into the interior section 93 of the implant 90. The distal section 21 may further be positioned at other locations relative to the implant 90 for placing fill material as needed.

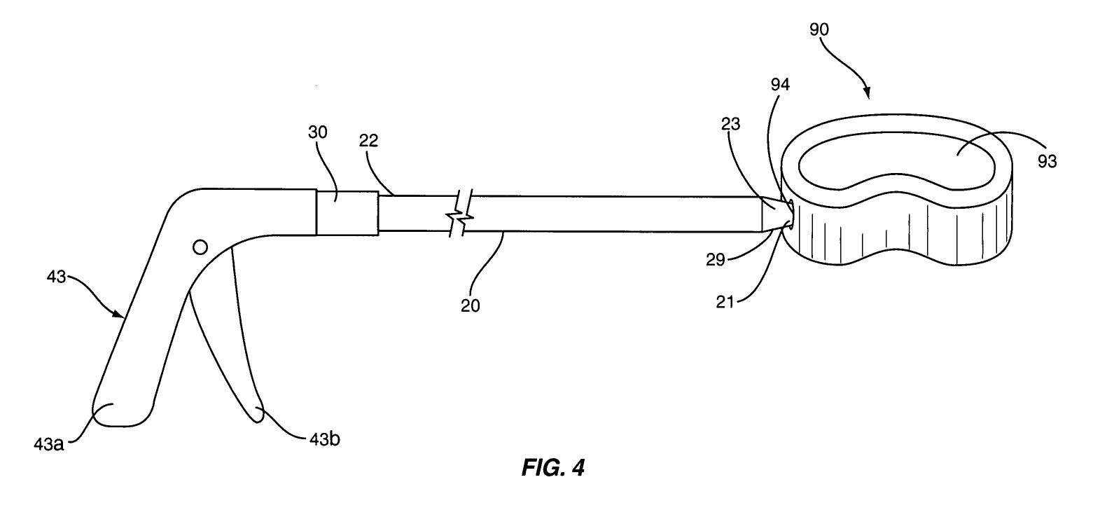

FIG. 4 illustrates another embodiment featuring a delivery device comprising an actuatable handle 43 providing delivery of the fill material. Handle 43 comprises first and second sections 43a, 43b that are relatively movable. Squeezing the sections 43a, 43b forces the fill material in the reservoir 30 to move through the insertion body 20 and out the distal end 21 to the implant 90. Each movement of the sections 43a, 43b may cause a predetermined amount of fill material to move from the distal end 21. In this manner, the surgeon is able to accurately determine the amount of fill material used with the implant 90. In another embodiment, sections 43a, 43b may have a variety of actuations points that each delivers a different amount of fill material. By way of example, a first amount of movement delivers a first amount of fill material, and a second greater amount of movement delivers a greater amount of fill material. Audible and/or tactile feedback may be used to determine the movements. In one embodiment, handle 43 is operatively connected to a pressure source that is able to move the fill material during actuation. In another embodiment, handle 43 is connected to a mechanism within the supply reservoir 30 that forces the fill material through the distal end 21.

The embodiment of FIG. 4 includes the insertion body 20 fixedly connected to the delivery device handle 43. The surgeon may grasp the handle 43 when placing the implant 90 within the body and also to deliver the fill material. The fill material may be loaded in the supply reservoir 30 through the distal end prior to the procedure, or through an opening in the insertion body 20 or handle 43. Distal end of the insertion body 20 includes a tapered section 29 that fits within an aperture 94 in the implant 90. The end is substantially flat to provide a contact surface for moving and positioning the implant 90 with the insertion body 20.

Use of the embodiment in FIG. 4 includes inserting the tapered section 29 into the aperture 94 and attaching the implant 90 to the insertion body 20. Once positioned, handle 43 is actuated to move the fill material from the supply reservoir 30 through the distal end 21 and to the implant 90. This may include filling an interior section 93 of the implant 90 and/or the exterior surfaces of the implant 90. Once complete, the tapered end 29 is removed from the aperture 94 leaving the implant 90 and fill material within the patient.

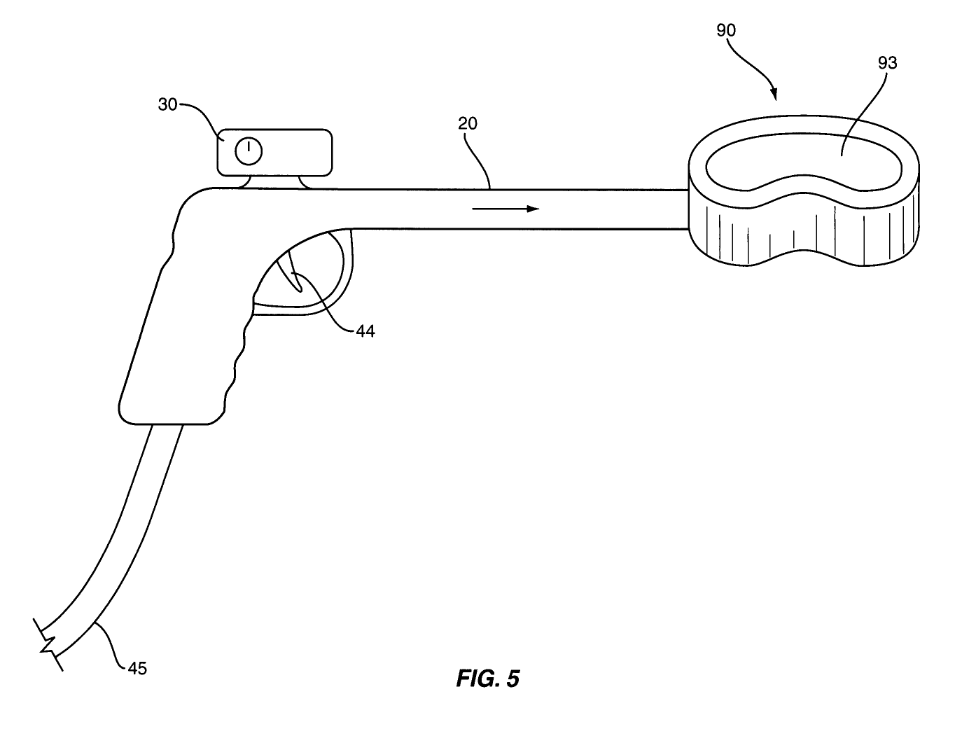

FIG. 5 illustrates another embodiment having a pressurized delivery source. A pressurized line 45 is operatively connected to the supply reservoir 30. A trigger 44 controls the pressure exerted to the supply reservoir 30 through the line 45. Varying degrees of actuation of the trigger 44 results in varying pressure amounts applied to the fill material and thus different flow rates being expelled from the distal end 21.

In another embodiment, insertion body 20 has an arcuate shape to facilitate insertion and placement of the implant 90. The entirety or a limited section or sections of the body 20 may be arcuate. Insertion body 20 may have a bent section again for facilitating insertion and placement. In one specific embodiment, the body has about a 90° bend. A variety of different insertion bodies 20 each having a different bend amount may be available to the surgeon prior to the procedure. The surgeon determines the amount of bend required and selects the appropriate body for the specific procedure. Two or more different bodies may be used during a procedure.

The embodiments illustrated and described are examples of the device and method. The elements of the various embodiments may be inchangeable with other elements described in other embodiments. By way of example, the external supply reservoir of FIG. 5 may be incorporated into the embodiment illustrated in FIG. 2.

The implant 90 may have a variety of different shapes and sizes. In one embodiment, implant 90 is substantially kidney shaped. In other embodiments, implant 90 has other shapes including circular, oval, polygonal, etc. In one embodiment, implant 90 is for use in intervertebral space between vertebral members. In other embodiments, implant 90 is positioned within other locations within the patient.

Implant 90 may be inserted into the patient in a variety of different approaches. In one embodiment, implant 90 is inserted in a substantially lateral approach. In other embodiments, implant 90 is inserted with posterior and anterior approaches.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A device to move fill material to an implant comprising:
an elongated rigid insertion body having a distal end spaced apart from a proximal end, the distal end having a threaded connection mechanism to attach directly to the implant;
a channel extending through the insertion body from the distal end to the proximal end;
a supply reservoir operatively connected to the channel to hold an amount of the fill material; and
a delivery device to move the fill material from the supply reservoir, through the channel, and out of the distal end to the implant.

2. The device of claim 1, wherein a section of the delivery device is sized to fit within and move through the proximal end of the insertion body to move the fill material from the supply reservoir through the distal end.

3. The device of claim 1, wherein the delivery device further comprises a handle to manipulate the connected insertion body and the implant.

4. The device of claim 1, wherein the supply reservoir is positioned distant from the channel.

5. The device of claim 1, further comprising a trigger mechanism to control the actuation of the delivery device.

6. The device of claim 1, further comprising a feedback mechanism operatively connected with the delivery device to limit a rate of the fill material that is delivered through the distal end.

7. The device of claim 1, further comprising a second supply reservoir operatively connected to the channel to hold a second amount of the fill material, the delivery device operatively connected to the second supply reservoir to move the fill material from the second supply reservoir, through the channel, and out of the distal end to the implant.

8. The device of claim 1, further comprising a spacer positioned between the delivery device and the distal end to move the fill material through the distal end.

9. A device to move fill material to an implant comprising:
an elongated rigid insertion body having a distal end and a spaced apart proximal end, the distal end having a connection mechanism to attach directly to the implant;
a channel extending through the insertion body from the distal end to the proximal end, the channel sized to contain the fill material; and
an elongated rigid delivery device having a first end and a second end, the first end sized to fit within the proximal end of the insertion body and fit through the channel and move the fill material within the channel out of the distal end of the insertion body and to the implant.

10. The device of claim 9, further comprising the proximal end of the insertion body and the first end of the delivery device being threaded for threadingly engaging the insertion body and the delivery device.

11. The device of claim 9, wherein the distal end of the insertion body has a tapered configuration that fits within an aperture in the implant.

12. The device of claim 9, further comprising a spacer positioned within the channel between the delivery device and the distal end, the spacer sized to move along the channel and move the fill material out of the distal end.

13. A method of placing an implant within a patient and moving fill material to the implant, the method comprising the steps of:
prior to insertion of the implant, placing fill material within a supply reservoir;
connecting a distal end of an insertion body to the implant;
manipulating the insertion body to position the implant within the patient;
while the insertion body is still attached to the implant, moving the fill material from the supply reservoir through the insertion body and to the implant; and
thereafter, disconnecting the insertion body from the implant and removing the insertion device from the patient.

14. The method of claim 13, wherein the step of connecting the distal end of the insertion body to the implant comprises threading the distal end onto the implant.

15. The method of claim 13, wherein the step of connecting the distal end of the insertion body to the implant comprises press fitting the distal end into an aperture in the implant.

16. The method of claim 13, wherein the step of manipulating the insertion body comprises connecting a delivery device to the insertion body and controlling movement of the implant and the insertion body through manipulations of the delivery device.

17. The method of claim 13, wherein the step of moving the fill material from the supply reservoir through the insertion body and to the implant comprises inserting and moving an instrument through a section of the insertion body and moving the fill material to the implant.

18. The method of claim 13, wherein the step of moving the fill material from the supply reservoir through the insertion body and to the implant comprises actuating a trigger on the delivery device to apply a pressure to the fill material in the supply reservoir.

19. The method of claim 13, wherein the step of moving the fill material from the supply reservoir through the insertion body and to the implant comprises actuating a handle with each actuation moving a predetermined amount of the fill material from the supply reservoir into the insertion device.

20. The method of claim 13, further comprising re-connecting the insertion body to the implant and moving additional fill material from the supply reservoir to the implant.

21. The method of claim 13, further comprising simultaneously moving the fill material to the implant and contacting the implant with the distal end to position the implant within the patient.

22. The method of claim 13, further comprising moving the fill material from a second supply reservoir through the insertion body and to the implant.

23. A method of placing an implant within a patient and moving fill material to the implant, the method comprising the steps of:
prior to insertion of the implant, placing fill material within a supply reservoir;
manipulating an insertion body connected to the supply reservoir and positioning the implant within the patient;
while positioning the implant, moving the fill material from the supply reservoir through the insertion body and to the implant; and
thereafter, removing the insertion device from the patient.

24. The method of claim 23, further comprising press fitting a distal end of the insertion body into an aperture in the implant.
